(19) United States
(12) Patent Application Publication
(10) Pub. No.: US 2022/0196557 A1
(43) Pub. Date: Jun. 23, 2022

(54) ANGULAR DEPTH RESOLVED RAMAN SPECTROSCOPY APPARATUS AND METHOD

(71) Applicant: CYTOVERIS INC., Farmington, CT (US)

(72) Inventors: Guoan Zheng, Vernon, CT (US); Alan Kersey, South Glastonbury, CT (US); Rishikesh Pandey, Unionville, CT (US); David Fournier, Northborough, MA (US)

(73) Assignee: CytoVeris Inc., Farmington, CT (US)

(21) Appl. No.: 17/601,631

(22) PCT Filed: Apr. 6, 2020
(86) PCT No.: PCT/US20/26864

§ 371 Date: Oct. 5, 2021

Related U.S. Application Data
(60) Provisional application No. 62/829,877, filed on Apr. 5, 2019.

(51) Int. Cl. G01N 21/65; G01N 21/64; G01J 3/44

(52) U.S. Cl. CPC G01N 21/65; G01J 2003/4424; G01J 3/4412; G01N 21/6486

(57) ABSTRACT

An apparatus and method for analyzing a tissue sample to provide depth-selective information includes at least one light source, collection light optics, and a light detector. The light source is configured to produce a light beam having one or more wavelengths of light that cause a tissue sample to produce Raman light signals upon interrogation of the tissue sample. The light beam is oriented to impinge on an exposed surface of the tissue sample at a point of incidence (POI), and oriented so that the light beam enters the tissue sample at an oblique angle relative to the exposed surface of the tissue sample. The collection light optics are configured to collect the Raman light signals emanating from the tissue sample at one or more predetermined lateral distances from the point of incidence. The light detector is configured to receive the Raman light signals from the collection light optics.

This application claims priority to U.S. patent application No. 62/829,877 filed Apr. 5, 2019, which is herein incorporated by reference in its entirety.

### BACKGROUND OF THE INVENTION

### 1. Technical Field

The present disclosure relates to systems and methods for the examining mammalian tissue using Raman spectroscopy, and more specifically systems and methods for the examining mammalian tissue using Raman spectroscopy at increased tissue depths.

### 2. Background Information

Raman spectroscopy is a chemical imaging technique that may be used to provide structural fingerprints of biomolecules. The chemical specificity of Raman spectroscopy originates from the interaction of light with the vibrational modes of the molecules being interrogated. In this regard, Raman spectroscopy requires no artificial modification of the sample and permits a comprehensive characterization of heterogeneous biological tissues. Conventional Raman systems, however, are limited to evaluating tissue only at or near the surface of a tissue sample.

What is needed is a system and methodology that enables Raman spectroscopy to be used to examine tissue at substantial depths below the surface of a tissue sample.

### SUMMARY

According to an aspect of the present disclosure, an apparatus for analyzing a tissue sample is provided. The apparatus includes at least one light source, collection light optics, and a light detector. The at least one light source is configured to produce a light beam having one or more wavelengths of light that cause a tissue sample to produce Raman light signals upon interrogation of the tissue sample by the one or more wavelengths of light. The light beam is oriented to impinge on an exposed surface of the tissue sample at a point of incidence (POI), and oriented so that the light beam enters the tissue sample at an oblique angle relative to the exposed surface of the tissue sample at the POI. The collection light optics are configured to collect the Raman light signals emanating from the tissue sample at one or more predetermined lateral distances from the point of incidence. The light detector is configured to receive the Raman light signals from the collection light optics.

In any of the aspects or embodiments described above and herein, the collection optics may include a light selection device configured to permit passage of the Raman light signals at only one of said predetermined lateral distances from the point of incidence.

In any of the aspects or embodiments described above and herein, the apparatus may include a linear actuator configured to laterally move the light selection device to permit passage of the Raman light signals at a first of the predetermined lateral distances or a second of the predetermined distances.

In any of the aspects or embodiments described above and herein, the light selection device may be a member having a confocal slit member or a member having a pin-hole aperture.

In any of the aspects or embodiments described above and herein, the apparatus may further include an analyzer in communication with the linear actuator and a memory device configured to store instructions, which instructions when executed cause the analyzer to control the linear actuator to move the light selection device to permit passage of said Raman light signals at only one of said predetermined lateral distances.

In any of the aspects or embodiments described above and herein, the light selection device may be controllable to permit passage of the Raman light signals at each of the predetermined lateral distances separately.

In any of the aspects or embodiments described above and herein, the light selection device may be a spatial light modulator or a digital micro-mirror device.

In any of the aspects or embodiments described above and herein, the apparatus may include an analyzer in communication with the light selection device and a memory device configured to store instructions, which instructions when executed cause the analyzer to control the light selection device to permit passage of the Raman light signals at each of the predetermined lateral distances separately.

In any of the aspects or embodiments described above and herein, the collection optics may include a light selection device configured to permit passage of the Raman light signals at only at a plurality of the predetermined lateral distances from the point of incidence concurrently.

In any of the aspects or embodiments described above and herein, the apparatus may include at least one optical fiber disposed to receive and transfer the light beam produced by the light source to the exposed surface of the tissue sample, the optical fiber having a lengthwise axis.

In any of the aspects or embodiments described above and herein, the optical fiber may include a canted end-face surface, which end-face surface is configured to cause light emanating from the optical fiber to exit at an angle divergent from the lengthwise axis of the optical fiber.

In any of the aspects or embodiments described above and herein, the optical fiber may include an end-face surface and a diffractive optical element attached to the end-face surface, the diffractive optical element configured to cause light emanating from the diffractive optical fiber to exit at an angle divergent from the lengthwise axis of the optical fiber.

In any of the aspects or embodiments described above and herein, the diffractive optical element may be configured to cause light at a first said wavelength emanating from the diffractive optical fiber to exit at a first angle divergent from the lengthwise axis of the optical fiber, and light at a second said wavelength emanating from the diffractive optical fiber to exit at a second angle divergent from the lengthwise axis of the optical fiber, the second angle different from the first angle. The apparatus may further include an analyzer in communication with the light source and a memory device configured to store instructions, which instructions when executed cause the analyzer to control the light source to selectively change said wavelength of light produced and thereby change said light divergent angle.

According to another aspect of the present disclosure, a method for analyzing a tissue sample is provided that includes: a) using a light source to produce a light beam having one or more wavelengths of light that cause a tissue sample to produce Raman light signals upon interrogation of the tissue sample by the one or more wavelengths of light, wherein the light beam is oriented to impinge on an exposed surface of the tissue sample at a point of incidence (POI), and oriented so that the light beam enters the tissue sample at an oblique angle relative to the exposed surface of the tissue sample at the POI; b) collecting first Raman light signals at a first predetermined lateral distance from the POI and transferring the first Raman light signals to a light detector configured to receive said first Raman light signals and produce first light detector signals representative of the first Raman light signals, and collecting second Raman light signals at a second predetermined lateral distance from the POI and transferring the second Raman light signals to the light detector configured to receive said second Raman light signals and produce second light detector signals representative of the second Raman light signals; and c) analyzing the first light detector signals to produce information regarding the tissue sample at a first position within the sample, the first position located at a first lateral distance from the POI and at a first depth distance from the exposed surface, and analyzing the second light detector signals to produce information regarding the tissue at a second position within the sample, the second position located at a second lateral distance from the POI and at a second depth distance from the exposed surface, wherein the second lateral distance is greater than the first lateral distance and the second depth distance is greater than the first depth distance.

In any of the aspects or embodiments described above and herein, the method may further include actuating a light selection device to permit passage of said Raman light signals at only the first predetermined lateral position or the second lateral position.

In any of the aspects or embodiments described above and herein, the method may further include actuating a light selection device to permit passage of said Raman light signals at only the first predetermined lateral position and the second lateral position.

In any of the aspects or embodiments described above and herein, the method may further include providing at least one optical fiber disposed to receive and transfer the light beam produced by the light source to the exposed surface of the tissue sample, the optical fiber having a lengthwise axis, the optical fiber including an end-face surface, and a diffractive optical element attached to the end-face surface, the diffractive optical element configured to cause light emanating from the diffractive optical fiber to exit at an angle divergent from the lengthwise axis of the optical fiber, and controlling the light source to selectively change said wavelength of light produced by the light source and thereby change said light divergent angle.

### BRIEF DESCRIPTION OF THE DRAWINGS

FIG. 1 is a diagrammatic view of a tissue sample being impinged by a light beam at an oblique angle.

FIG. 2 is a generic diagram of certain system embodiments according to the present disclosure.

FIG. 3 is a diagrammatic view of a system embodiment.

FIG. 4 is a diagrammatic view of a system embodiment.

FIG. 5 is a diagrammatic view of a system embodiment.

FIG. 6 is a diagrammatic view of a system embodiment.

FIG. 7 is a diagrammatic view of an input fiber and collection fiber embodiment.

FIG. 8 is a diagrammatic view of an input fiber and collection fiber embodiment.

FIG. 9 is a diagrammatic view of a system embodiment.

FIG. 10 is a diagrammatic view of a system embodiment.

### DETAILED DISCLOSURE

The present disclosure includes apparatus and methods that utilize an imaging technique that may be referred to as “angular depth resolved Raman spectroscopy” or “ADRRS”, to get Raman spectral information of a three-dimensional (“3D”) object at different depths from the surface of the tissue sample. The present disclosure apparatus and method may be utilized to analyze/image an ex-vivo tissue sample or an in-vivo tissue sample.

Light incident to any material has a certain probability of being scattered. As will be explained below, the present disclosure advantageously provides a means for sensing Raman light scattering characteristics of certain materials at significant subcutaneous depths. When photons are scattered, most of them are elastically scattered, and that the scattered photons have the same energy (e.g., frequency, wavelength, color) as the incident photons but different directions. This type of photon scattering is typically referred to as “Rayleigh scattering”. Raman scattering, in contrast, refers to inelastic scattering where there is an exchange of energy and a change in the light's direction. All materials exhibit Raman scattering in response to incident light. The Raman spectrum for a given material (including those found in tissue) typically complex due to the variety of molecular vibrations present within the material, and the material is identifiable based on the Raman spectrum. An exemplary Raman spectrum may include a number of different peaks at a certain wavelengths or ‘wavenumber’ offsets from incident light, which are uniquely characteristic of the material. Hence, the Raman spectrum of a particular material can be thought of as a “fingerprint” of that particular material, and can be used for identification purposes.

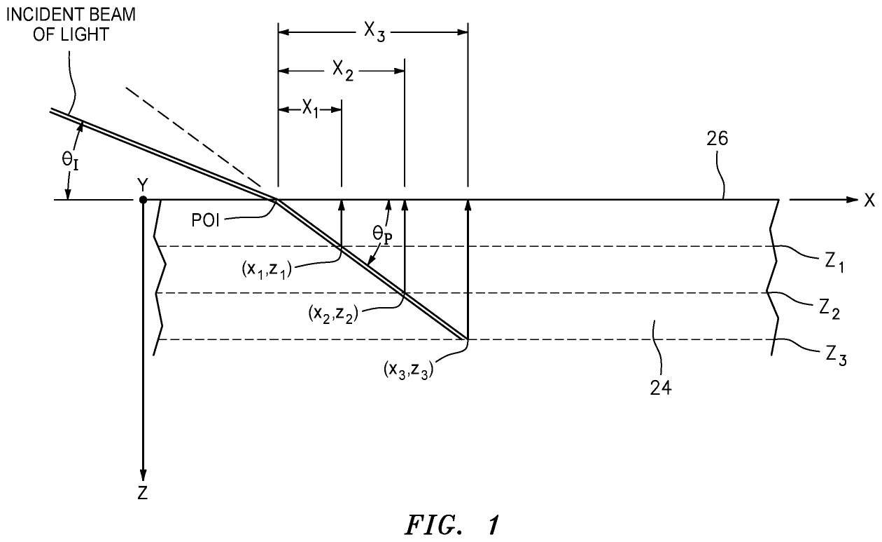

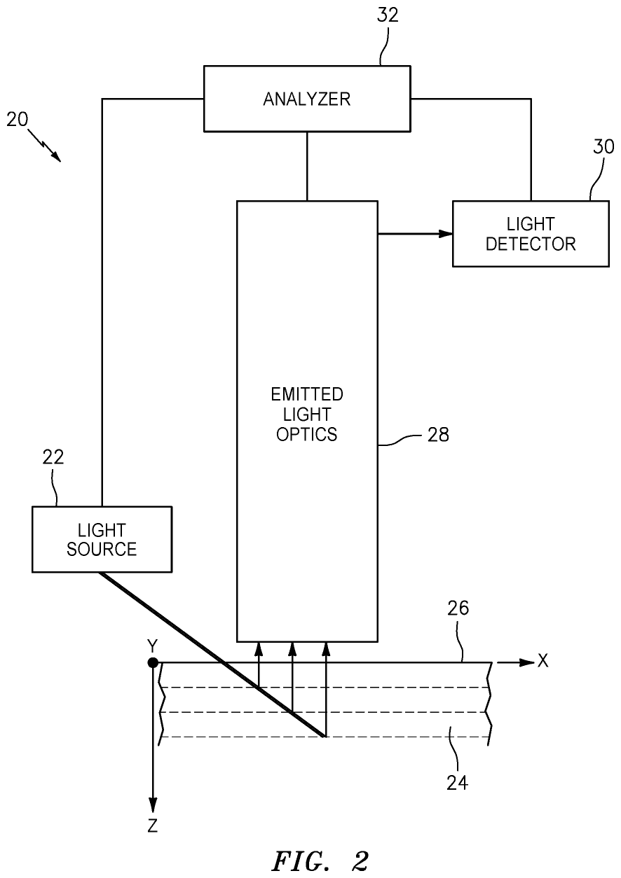

Aspects of the present disclosure system 20 include a light source 22 that directly or indirectly produces a beam of light to illuminate a 3D tissue sample 24. The light source 22 is oriented so that the beam of light is incident to the surface of the tissue sample at an oblique angle (i.e., an acute angle), and thereafter propagates through the 3D tissue sample 24 at an oblique angle. Due to differences in refractive index, the oblique angle of the light beam propagating within the tissue sample (e.g., see “Θ_{P}” in FIG. 1) will shift from the oblique angle of the incident light beam (e.g., see “Θ_{I}” in FIG. 1), but will still be at an oblique angle. The shift in oblique angle between the incident light beam and the propagating light beam is known and accounted for within the present disclosure. To facilitate the description herein, the oblique angle of the light beam (regardless of whether it is the angle of the incident light beam, or the propagating light beam) will be generically be referred to as “oblique” (and shown as a single angle in the Figures), with the understanding that differences in refractive index may shift the oblique angle between the incident light beam and the propagating light beam to some degree. In some embodiments, incident light beam may be dithered (i.e., rapidly scanned) along a Y-axis to form a light sheet, where the Y-axis is perpendicular to an X-Y plane (e.g., see FIG. 1, where the Y-axis is perpendicular to the plane of the Figure). As will be described herein, a beam of light that is transmitted through a 3D tissue sample 24 at an oblique angle provides improved light beam penetration depth into the 3D tissue sample 24, and therefore an improved ability to generate Raman spectroscopy data at deeper depths in the tissue sample 24.

As shown diagrammatically in FIG. 1, an incident light beam transmits through the surface 26 of a 3D tissue sample 24 at a point of incidence (“POI”). The light beam is oriented at an oblique incident angle theta (“Θ_{P}”) relative to the tissue sample surface 26. The light beam may be described as traveling within the tissue sample 24 along both an X-axis (i.e., a lateral distance along the surface of the tissue sample) and along a Z-axis (i.e., a depth from the surface of the tissue sample), wherein the POI may be considered to be the origin of the aforesaid axes. Hence, as the obliquely oriented light beam travels into the tissue sample 24, tissue located at different spatial positions within the sample is interrogated by the light beam; i.e., tissue at lateral distances X and depth positions Z—shown in FIG. 1 as positions (X₁, Z₁), (X₂, Z₂), and (X₃, Z₃) within the tissue sample, where X₃>X₂>X₁ and Z₃>Z₂>Z₁. As the incident light beam interrogates the tissue, Raman signals are produced in the manner described above from tissue constituents such as cells and extracellular matrix, other biological material such as calcium deposits—which are hallmarks of microcalcification in breast tissue—in response to the interrogating light. The Raman signals produced are therefore specific to the tissue located at those spatial locations (X₁, Z₁), (X₂, Z₂), and (X₃, Z₃). The aforesaid Raman signals can be sensed at the surface 26 of the tissue sample 24 at the respective lateral positions. The present disclosure, therefore provides a means for collecting Raman spectroscopic information from tissue located within a 3D tissue sample 24 at different depths therein.

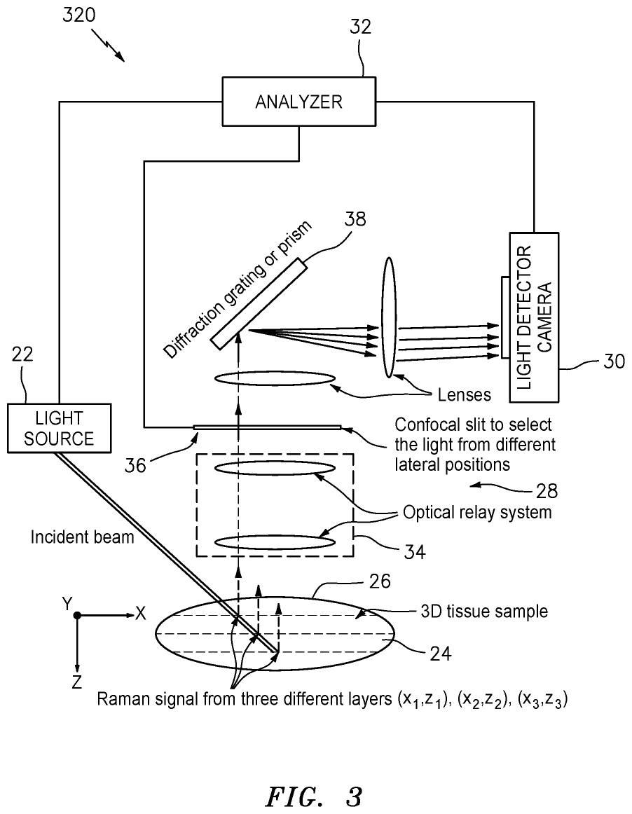

Embodiments of the present disclosure system 20 include at least one light source 22, collection light optics 28, and at least one light detector 30 (system shown diagrammatically in FIG. 3). Some system 20 embodiments include an analyzer 32. As will be described herein, the present disclosure contemplates a variety of different system 20 embodiments. The system embodiments described herein may refer to various different system components as being independent components. In alternative embodiments, system components otherwise described as independent may be combined, or arranged in a different manner than that shown in the Figures, or may be utilized with additional components, or different combinations of the components may be used, and still be within the scope of the present disclosure. The specific system 20 embodiments described herein are non-limiting examples of the present disclosure provided to illustrate aspects of the present disclosure, and are not intended to limit the present disclosure.

The light source 22 is configured to produce light, typically in predetermined wavelengths. In some embodiments, the light source 22 itself may be configured to produce an incident beam of light. In some embodiments, light produced by the light source 22 may be optically manipulated to produce an incident beam of light. Non-limiting examples of an incident beam that can be used include a regular Gaussian beam, a non-diffracting Bessel beam, an Airy beam, and a lattice light sheet. A light source 22 such as a Bessel beam that produces an incident beam with “self-healing” propagation properties is particularly useful because the light beam is typically able to penetrate deeper into tissue specimens. The light source 22 may provide incident light to a tissue sample 24 via free space or via elements (e.g., optical fibers) that provide a conduit for light produced by the light source 22 to travel to the tissue sample 24.

The collection light optics 28 are configured to collect, transfer, and/or process Raman signal scattered from the tissue sample 24 as a result of the light beam interrogation. The collection light optics 28 may include one or more lenses, filters, one or more light selection devices (e.g., a dichroic mirror, a confocal slit, a pinhole, a digital micro-mirror device, a spatial light modulators (SLM), a multi-apertured mask, and the like) for processing the received light and transferring it to a light detector. In some embodiments, scattered light received at a tissue sample surface 26 may be collected at the tissue sample surface and transferred by an optical relay system to other collection light optic components located remote from the point of collection at the skin; e.g., collected at the skin surface by optical fibers or fiber optic bundles, which may include filters or the like, and transferred to other collection light optics located remote from the tissue sample 24. Collection fibers of an ADRRS fiber probe according to the present disclosure may include a coating on the tip of each fiber to allow transmission of certain wavelengths or spectral range.

The light detector 30 is configured to receive light (e.g., Raman spectra) scattered from the interrogated tissue via the collection light optics 28 and produce signals representative thereof. The light detector 30 is configured for communications with an analyzer 32 (and/or a memory storage device) and produces signals that are in a form to be received by the analyzer 32 (and/or a memory storage device). As will be described herein, the present disclosure contemplates that light detector signals may be directly communicated to an analyzer 32 (locally or remotely located), or may be stored in a memory device and subsequently transferred to an analyzer 32. Non-limiting examples of light detectors 30 include light sensors that convert light energy into an electrical signal such as a photodiode, or a charge couple device (CCD), or a camera (e.g., a CMOS camera), or an array camera, or other photometric detectors known in the art.

The analyzer 32 is in communication with other components within the system, such as the at least one light source 22, the at least one light detector 30, the collection light optics 28, and the like, to control and or receive signals therefrom to perform the functions described herein. The analyzer 32 may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to perform the described method steps and/or to enable the system to accomplish the same algorithmically and/or coordination of system components. The analyzer 32 may include a single memory device or a plurality of memory devices. The present disclosure is not limited to any particular type of memory device, and may include read-only memory, random access memory, volatile memory, non-volatile memory, static memory, dynamic memory, flash memory, cache memory, and/or any device that stores digital information. The analyzer 32 may include, or may be in communication with, an input device (not shown) that enables a user to enter data and/or instructions, and may include, or be in communication with, an output device (not shown) configured, for example to display information (e.g., a visual display or a printer), or to transfer data, etc. Communications between the analyzer 32 and other system components (e.g., the light source 22, light detector 30, etc.) may be via a hardwire connection or via a wireless connection.

Diagrammatic illustrations of exemplary system embodiments according to the present disclosure are shown in FIGS. 3-10.

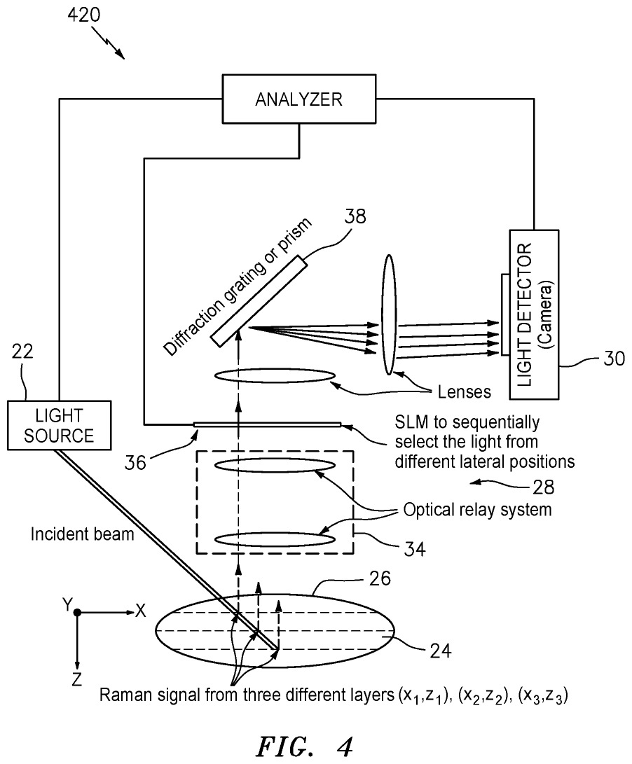

Referring to FIGS. 3 and 4, exemplary system embodiments 320, 420 that utilize ADRRS are shown. Each system includes a light source 22, collection light optics 28, a light detector 30, and an analyzer 32. The light source 22 produces an incident light beam interrogating the tissue sample surface at an oblique angle relative to the tissue sample surface 26. As stated above, a variety of different incident light beam configurations may be used; e.g., a regular Gaussian beam, a non-diffracting Bessel beam, an Airy beam, or the like. In some embodiments, the incident beam may be dithered (i.e., rapidly scanned) along the Y-axis shown in FIGS. 3 and 4 (perpendicular to the X-Z plane of the Figure) to form a light sheet. The obliquely applied light beam travels within the tissue sample in a direction having both X-axis and Z-axis components, and thereby interrogates tissue located at different spatial positions; i.e., tissue at lateral distances X and depth positions Z noted as (X₁, Z₁), (X₂, Z₂), and (X₃,Z₃). In both embodiments, the collection light optics includes an optical relay system 34 (e.g., including a plurality of lenses), a light selection device 36, a diffraction grating or prism 38, and one or more lenses and/or filters configured to manipulate the collected scattered light into a desirable form (e.g., a focused form, a columnar form, etc.). In the system embodiment shown in FIG. 3, the light selection device 36 is a confocal slit that is laterally moveable (e.g., along the X-axis). In the system embodiment shown in FIG. 4, the light selection device 36 is a spatial light modulator (SLM). The system embodiments shown in FIGS. 3 and 4 both include a light detector 30 (e.g., a camera) configured to receive the collected Raman light and produce signals representative thereof.

In the operation of the system embodiments of FIGS. 3 and 4, the obliquely oriented incident light beam travels within the tissue sample 24 along both an X-axis and a Z-axis, and thereby interrogates tissue located at different spatial positions; i.e., tissue at lateral and depth positions noted as (X₁, Z₁), (X₂, Z₂), and (X₃,Z₃). In response to the interrogating light beam, tissue at the respective lateral and depth positions produces Raman signals specific to the tissue located at the aforesaid positions. At least some of those Raman photons travel to the tissue sample surface at lateral positions aligned with the lateral position of the tissue producing the Raman signals. In the system embodiment shown in FIG. 3, the X-axis translatable confocal slit (or pinhole, etc.) is laterally positioned to receive the Raman signal light at particular lateral positions; e.g., in a first position, the confocal slit is laterally positioned to receive the Raman signal light scattered from the interrogated tissue located at spatial location (X₁, Z₁); in a second position, the confocal slit is laterally positioned to receive the Raman signal light scattered from the interrogated tissue located at spatial location (X₂, Z₂); in a third position, the confocal slit is laterally positioned to receive the Raman signal scattered from the interrogated tissue located at spatial location (X₃,Z₃), etc. In this manner, the system is configured to receive information (i.e., Raman signals) from the tissue sample 24 at multiple different depths (Z₁, Z₂, and Z₃, wherein Z₃>Z₂>Z₁). The lateral positioning of the confocal slit may be accomplished by a linear motor or the like controlled by instructions stored within the analyzer 32. In the system embodiment shown in FIG. 4, the SLM (or similar device such as a digital micro-mirror device, etc.) is configured to receive the Raman signals at particular lateral positions without physical translation of the entire device; e.g., in a first position, SLM is controlled to receive the Raman signal scattered from the interrogated tissue located at spatial location (X₁, Z₁); in a second position, the SLM is controlled to receive the Raman light scattered from the interrogated tissue located at spatial location (X₂, Z₂); in a third position, the SLM is controlled to receive the Raman signal scattered from the interrogated tissue located at spatial location (X₃,Z₃), etc. The operation of the SLM (or similar device such as a digital micro-mirror device, etc.) may be described as laterally sweeping to collect the Raman signal produced at different lateral positions (and therefore associated tissue sample depths). The operation of the SLM may be pursuant to instructions stored within the analyzer 32.

The Raman signal light selected by the light selection device 36 (e.g., confocal slit, pinhole, SLM, digital micro-mirror device, etc.) subsequently passes through additional optics (e.g., a lens, or the like) and then to a diffraction grating or a prism 38. The relative positioning of the optics (e.g., lens) and the diffraction grating/prism 38 may be chosen to optimize transfer of the Raman signal light; e.g., the diffraction grating/prism 38 may be placed at the pupil plane of the preceding lens. The diffraction grating/prism 38 reflects the Raman signal light towards the light detector 30. Light reflected from the diffraction grating/prism 38 may pass through optics (e.g., a lens or other device to orient the light in a desirable configuration) prior to impingement onto the detector 30. The light detector 30 receives the Raman signal light and produces signals representative thereof. The signals produced by the light detector 30 may be transferred to the analyzer 32, which may produce analytical data based on the aforesaid signals, or to a storage device for subsequent analysis. Some embodiments of the present system may be configured to obviate the use of a diffraction grating/prism 38; e.g., a light detector 30 directly aligned. In some system embodiments, at least one optical filter can be used to filter out Raman light directly and analyzed by a light detector 30.

FIG. 5 diagrammatically illustrates another system embodiment 520 that utilizes ADRRS. The system embodiment 520 shown in FIG. 5 includes a light source 22 and an analyzer 32 similar to or the same as described above (the analyzer 32 may be integrally included within the spectrometer 40). In the system embodiment 520 of FIG. 5, the collection light optics 28 includes an optical relay system 34 the same as or similar to that described above. In the system embodiment 520 of FIG. 5, the light selection device 36 includes a multi-pin-hole array (or a multi-aperture “mask”, or the like) having apertures laterally positioned to receive the Raman signal light at particular lateral positions without physical translation of the entire device; e.g., one or more first apertures positioned to receive Raman signal light scattered from the interrogated tissue located at spatial location (X₁, Z₁); one or more second apertures positioned to receive Raman signal scattered from the interrogated tissue located at spatial location (X₂, Z₂); one or more third apertures positioned to receive Raman signal scattered from the interrogated tissue located at spatial location (X₃,Z₃), etc. In this manner, the system 520 is configured to receive information (i.e., Raman signals) from the tissue sample 24 at multiple different depths (Z₁, Z₂, and Z₃, wherein Z₃>Z₂>Z₁). In alternative embodiments, rather a multi-pin-hole array / multi-aperture mask, the system 520 may include an array of optical fibers (not shown) arranged to receive Raman signal light at aforesaid lateral positions (e.g., X₁, X₂, X₃, etc.). The apertures of the light selection device 36 (or the optical fibers) may be coupled to the input plane of a spectrometer 40 to spatially separate the respective input Raman signal. FIG. 5 includes an expanded view of a non-limiting spectrometer 40 example having a diffraction grating 38 and a light detector 30 in the form of a camera array. In this exemplary spectrometer 40, the spatially separated Raman signal light inputs may impinge on different rows of the camera array. The signals produced by the camera array for each spatially separated input may be distinguished from one another for subsequent processing to provide tissue sample information at the respective tissue depths. This system embodiment can be used to produce simultaneous monitoring of the Raman signal from each lateral position/tissue depth (X, Z) of the tissue sample.

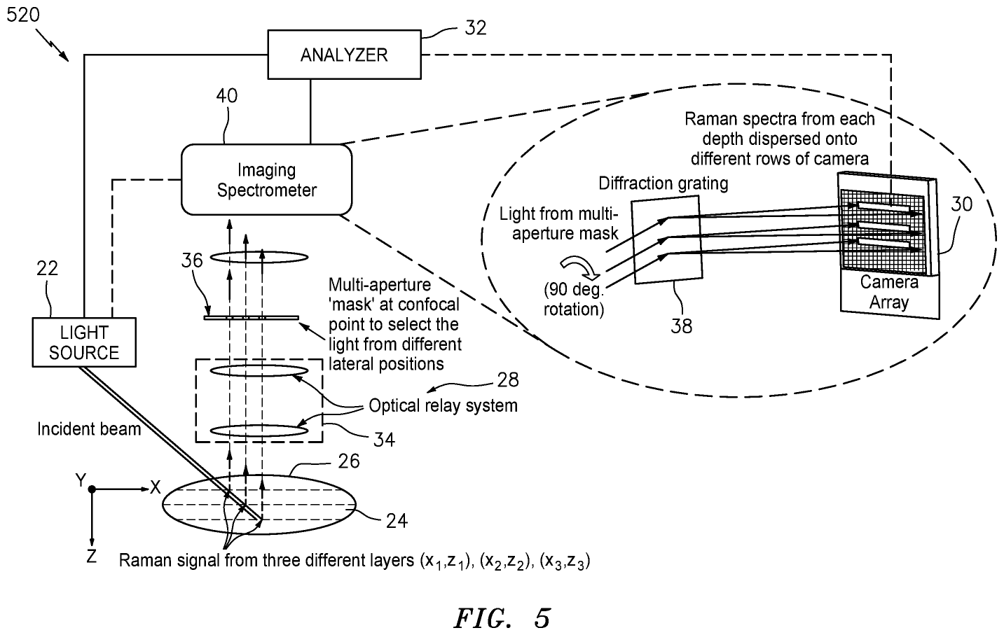

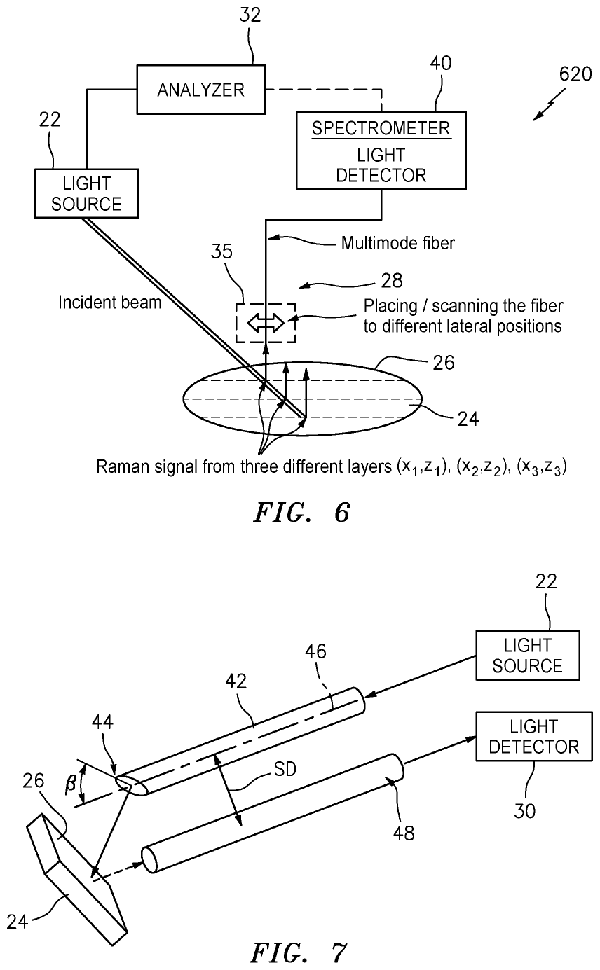

FIG. 6 diagrammatically illustrates another system embodiment 620 that utilizes ADRRS. The system embodiment shown in FIG. 6 includes a light source 22. The system embodiment 620 may include a spectrometer 40 and an analyzer 32 similar to or the same as described above (the analyzer 32 may be included in the spectrometer 40). In this embodiment, the collection light optics 28 may include one or more optical fibers (“detection fibers”) that can be selectively positioned at different lateral positions (e.g., along the X-axis) relative to the point of the light incidence by the light beam; e.g., by an actuating system 35 configured to more the fibers laterally. By positioning the optical fiber(s) at different lateral positions, the Raman signal can be detected at different depths of the tissue sample 24. The Raman signal light may be passed via the optical fiber(s) to system components such as those described herein (e.g., a spectrometer 40 having a diffraction grating/prism 38, a light detector 30, and an analyzer 32), optics such as lens, filters, etc. This system embodiment 620 is not limited to use with a spectrometer 40 and may be used with independent elements such as a diffraction grating 38, a light detector 30, analyzer, analyzer 32, optical filters etc.

In those system embodiments wherein a beam of light is used that is obliquely incident to the surface of the 3D tissue sample (e.g., applied to the surface at an oblique angle “Θ_{I}”) and propagates within the tissue sample at an oblique angle (e.g., “Θ_{P}”—See FIG. 1), the present disclosure is not limited to any particular oblique angle. As described herein, the present disclosure system embodiments can be used to recover the Raman spectra associated with tissue located at different depths within the tissue sample. The magnitude of the oblique angle may be varied to change the depth of tissue interrogated at respective lateral positions.

The above-described system embodiments detail a light source that is oriented to produce a beam of light that is incident to the surface of the 3D tissue sample at an oblique angle. The aforesaid oblique light beam orientation may be accomplished by a fixture that holds the light source 22 (or a portion of it, or a conduit for the light produced by the light source, etc.) in an oblique orientation. The present disclosure is not, however, limited to any specific mechanism for producing the obliquely oriented light beam. For example, in an alternative embodiment shown in FIG. 7, the light source 22 may be in communication with one or more optical fibers 42 (i.e., “input fibers 42”), with each input fiber 42 having a canted end-face surface 44, preferably polished, that is disposed at a non-perpendicular angle (“β”) relative to the lengthwise axis 46 of the optical fiber 42. A light beam exiting the canted end-face surface 44 exits perpendicular to the end-surface, and therefore at an angle to the lengthwise axis 46 of the input fiber 42 (i.e., complimentary to the angle β of the end-surface). The input optical fiber(s) 42 are positioned relative to the surface 26 of the tissue sample 24 to produce the incident light beam at an oblique angle as described above. In the embodiments shown in FIG. 7, the system 20 may include one or more optical fibers 48 (“collection fibers 48”) separated from the input fiber(s) 42 by predetermined distances. In FIG. 7, the input fiber 42 is shown extending substantially parallel to a collection fiber 48 by a separation distance “SD”. In alternative embodiments, additional collection fibers 48 may be spaced apart from one another by uniform distances (e.g., 1 SD, 2 SD, 3 SD, etc.) or the collection fibers 48 may be separated by different separation distances. These input and collection fibers 42, 48 can form part of probe assembly or configuration. Since an oblique incident light beam travels at deeper tissue depths in the lateral direction, the Raman signals captured by a collection fiber(s) 48 in close proximity to the input fiber(s) 42 can be used to interrogate very shallow tissue depths contiguous with the surface 26 of the tissue sample 24 (e.g., at the top one to two hundred micrometers (100 μm-200 μm) of the sample); e.g., using input fibers 42 having a canted end-surface 44 produced by strongly angle polishing the tip of the input fiber 42.

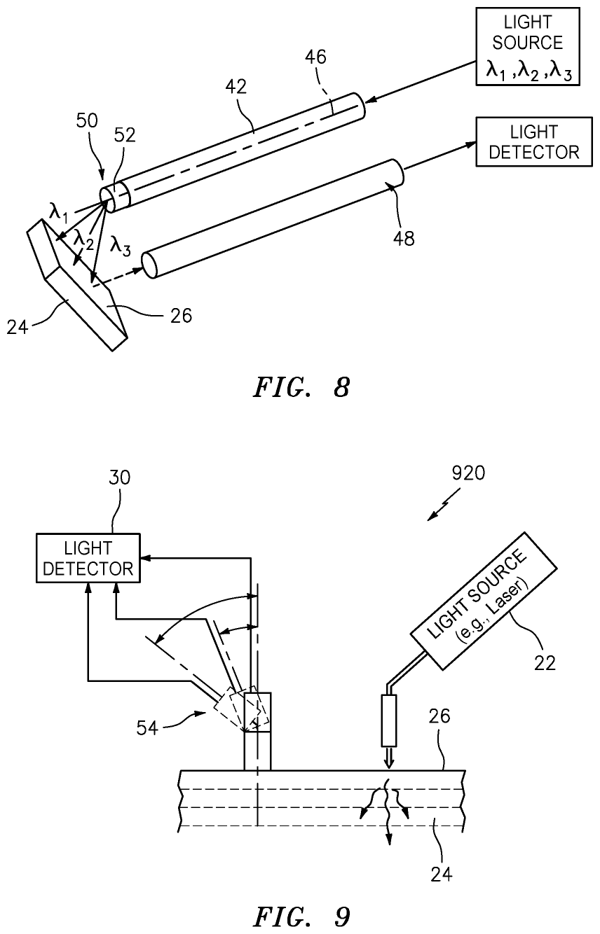

FIG. 8 illustrates a further alternative embodiment wherein the light source is in communication with one or more optical fibers 42 (i.e., “input fibers 42”), with each input fiber 42 having a diffractive optical element 50 coupled to or bonded to the end surface 52 of the input fiber 42. The fiber end surface 52 may be perpendicular to the lengthwise axis 46 of the fiber 42, or the fiber end surface 52 may be canted at an angle (i.e., non-perpendicular) to the lengthwise axis 46 of the fiber 42. Light passing through the diffractive optical element 50 is subjected to an angular offset. If the end surface 52 of the input fiber 42 is canted, the diffractive optical element 50 can add an additional angular offset to the direction of the light beam. In addition, by changing the wavelength of the light that forms the light beam, the angular offset produced by the diffractive optical element 50 can be modulated or controlled, and therefore the angle of incident light relative to the tissue sample surface 26 can be modulated or changed. An analyzer 32 may be configured to control the light source to produce different wavelengths of light and therefore the angular offset of the light beam exiting the diffractive optical element 50. A collection fiber 48 offset from the incident light beam impingement position will then receive Raman signatures from differing depths in the tissue dependent on the excitation wavelength. A plurality of collection fibers 48 at different offset positions could be used to collect the produced Raman signal light.

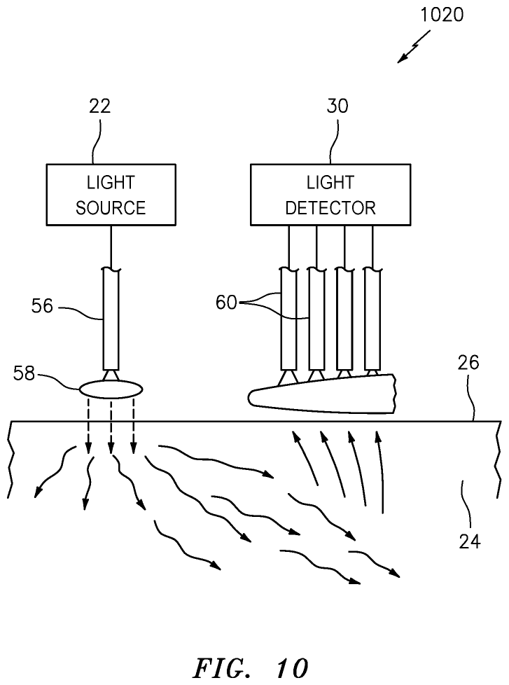

FIG. 9 illustrates a further alternative system embodiment 920 wherein the system 920 is configured such that the light beam from a light source 22 (e.g., a laser) is in a substantially normal orientation to the surface 26 of the tissue sample 24 (e.g., at about a right angle). In this embodiment, the angle at which the Raman signals are detected from the surface 26 of the tissue sample 24 may be varied and therefore depth sensitivity (e.g., acquiring Raman signals generated at different tissue depths) is attained. A rotational lens structure 54 is an example of a light collecting structure that may be used to vary the angle at which the Raman signals are detected from the surface 26 of a tissue sample 24. A specific example of a rotational lens structure 54 is a gradient index lens (often referred to as a “GRIN lens”). This alternative ADRRS embodiment may be referred to as an inverse of the above embodiments wherein the angle of the light source 22 is oblique to create the tissue sample depth information via the Raman signals.

FIG. 10 illustrates a further alternative system 1020 embodiment wherein the system 1020 is configured such that a beam of light from a light source 22 (e.g., a laser) is disposed to impinge the surface 26 of a tissue sample 24 at a substantially normal orientation to the surface 26 of the tissue sample 24 (e.g., at about a right angle). In the diagram of FIG. 10, the light source 22 is shown in communication with an optical fiber 56 that functions as a conduit for the light beam, and an optical element 58 (e.g., a lens) is shown disposed between the source optical fiber 56 and the surface 26 of the tissue sample 24. Neither of the optical fiber 56 or the optical element 58 are required. This embodiment utilizes a plurality of light collection elements 60 (e.g., optical fibers, typically all having a common diameter) and an optical element 62 (e.g., a lens) disposed between the collection elements 60 and the surface 26 of the tissue sample 24. The optical element 62 is configured to impart a different angular acceptance angle for each of the light collection elements 60. As a result, the Raman signal light collected by each of the collection elements 60 represents Raman signals scattered from tissue matter located at different tissue sample depths.

The present disclosure includes methodologies for operating the system embodiments described above.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. A coupling between two or more entities may refer to a direct connection or an indirect connection. An indirect connection may incorporate one or more intervening entities or a space/gap between the entities that are being coupled to one another.

Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. An apparatus for analyzing a tissue sample in both ex-vivo and in-vivo conditions, comprising:
at least one light source configured to produce a light beam having one or more wavelengths of light that cause a tissue sample to produce Raman light signals upon interrogation of the tissue sample by the one or more wavelengths of light;
wherein the light beam is oriented to impinge on an exposed surface of the tissue sample at a point of incidence (POI), and oriented so that the light beam enters the tissue sample at an oblique angle relative to the exposed surface of the tissue sample at the POI;
collection light optics configured to collect said Raman light signals emanating from the tissue sample at one or more predetermined lateral distances from the point of incidence; and
a light detector configured to receive said Raman light signals from the collection light optics.

2. The apparatus of claim 1, wherein the collection optics includes a light selection device configured to permit passage of said Raman light signals at only one of said predetermined lateral distances from the point of incidence.

3. The apparatus of claim 2, further comprising a linear actuator configured to laterally move the light selection device to permit passage of said Raman light signals at a first of the predetermined lateral distances or a second of the predetermined distances.

4. The apparatus of claim 3, wherein the light selection device is a member having a confocal slit member or a member having a pin-hole aperture.

5. The apparatus of claim 3, further comprising an analyzer in communication with the linear actuator and a memory device configured to store instructions, which instructions when executed cause the analyzer to control the linear actuator to move the light selection device to permit passage of said Raman light signals at only one of said predetermined lateral distances.

6. The apparatus of claim 2, wherein the light selection device is controllable to permit passage of said Raman light signals at each of the predetermined lateral distances separately.

7. The apparatus of claim 5, wherein the light selection device is a spatial light modulator or a digital micro-mirror device.

8. The apparatus of claim 6, further comprising an analyzer in communication with the light selection device and a memory device configured to store instructions, which instructions when executed cause the analyzer to control the light selection device to permit passage of said Raman light signals at each of the predetermined lateral distances separately.

9. The apparatus of claim 1, wherein the collection optics includes a light selection device configured to permit passage of said Raman light signals at only at a plurality of the predetermined lateral distances from the point of incidence concurrently.

10. The apparatus of claim 9, wherein the light selection device is a multi-apertured mask or a multi-pin-hole array.

11. The apparatus of claim 1, further comprising at least one optical fiber disposed to receive and transfer the light beam produced by the light source to the exposed surface of the tissue sample, the optical fiber having a lengthwise axis.

12. The apparatus of claim 11, wherein the optical fiber includes an canted end-face surface, which end-face surface is configured to cause light emanating from the optical fiber to exit at an angle divergent from the lengthwise axis of the optical fiber.

13. The apparatus of claim 11, wherein the optical fiber includes an end-face surface and a diffractive optical element attached to the end-face surface, the diffractive optical element configured to cause light emanating from the diffractive optical fiber to exit at an angle divergent from the lengthwise axis of the optical fiber.

14. The apparatus of claim 13, wherein the diffractive optical element is configured to cause light at a first said wavelength emanating from the diffractive optical fiber to exit at a first angle divergent from the lengthwise axis of the optical fiber, and light at a second said wavelength emanating from the diffractive optical fiber to exit at a second angle divergent from the lengthwise axis of the optical fiber, the second angle different from the first angle; and
wherein the apparatus further comprises an analyzer in communication with the light source and a memory device configured to store instructions, which instructions when executed cause the analyzer to control the light source to selectively change said wavelength of light produced and thereby change said light divergent angle.

15. The apparatus of claim 1, wherein the at least one light source is configured to produce one or more of a regular Gaussian beam, a non-diffracting Bessel beam, an Airy beam, or a lattice light sheet.

16. A method for analyzing a tissue sample, comprising:
using a light source to produce a light beam having one or more wavelengths of light that cause a tissue sample to produce Raman light signals upon interrogation of the tissue sample by the one or more wavelengths of light, wherein the light beam is oriented to impinge on an exposed surface of the tissue sample at a point of incidence (POI), and oriented so that the light beam enters the tissue sample at an oblique angle relative to the exposed surface of the tissue sample at the POI;
collecting first Raman light signals at a first predetermined lateral distance from the POI and transferring the first Raman light signals to a light detector configured to receive said first Raman light signals and produce first light detector signals representative of the first Raman light signals, and collecting second Raman light signals at a second predetermined lateral distance from the POI and transferring the second Raman light signals to the light detector configured to receive said second Raman light signals and produce second light detector signals representative of the second Raman light signals; and
analyzing the first light detector signals to produce information regarding the tissue sample at a first position within the sample, the first position located at a first lateral distance from the POI and at a first depth distance from the exposed surface, and analyzing the second light detector signals to produce information regarding the tissue at a second position within the sample, the second position located at a second lateral distance from the POI and at a second depth distance from the exposed surface, wherein the second lateral distance is greater than the first lateral distance and the second depth distance is greater than the first depth distance.

17. The method of claim 16, further comprising actuating a light selection device to permit passage of said Raman light signals at only the first predetermined lateral position or the second lateral position.

18. The method of claim 16, further comprising actuating a light selection device to permit passage of said Raman light signals at only the first predetermined lateral position and the second lateral position.

19. The method of claim 16, further comprising providing at least one optical fiber disposed to receive and transfer the light beam produced by the light source to the exposed surface of the tissue sample, the optical fiber having a lengthwise axis, the optical fiber including an end-face surface, and a diffractive optical element attached to the end-face surface, the diffractive optical element configured to cause light emanating from the diffractive optical fiber to exit at an angle divergent from the lengthwise axis of the optical fiber; and
controlling the light source to selectively change said wavelength of light produced by the light source and thereby change said light divergent angle.

20. The method of claim 15, wherein the at least one light source is configured to produce one or more of a regular Gaussian beam, a non-diffracting Bessel beam, an Airy beam, or a lattice light sheet.
